(19) United States
(12) Patent Application Publication
(10) Pub. No.: US 2024/0108772 A1
(43) Pub. Date: Apr. 4, 2024

(54) AEROSOL REACHABLE AREA ESTIMATION SYSTEM, AEROSOL REACHABLE AREA ESTIMATION METHOD, AND NON-TRANSITORY COMPUTER-READABLE RECORDING MEDIUM

(71) Applicant: Panasonic Intellectual Property Management Co., Ltd., Osaka (JP)

(72) Inventors: TATSUSHI OHYAMA, Gifu (JP); MARIKO MIYASHITA, Hyogo (JP)

(21) Appl. No.: 18/539,415

(22) Filed: Dec. 14, 2023

Related U.S. Application Data
(63) Continuation of application No. PCT/JP2022/025776, filed on Jun. 28, 2022.

(30) Foreign Application Priority Data
Jul. 9, 2021 (JP) 2021-114109

(51) Int. Cl. A61L 2/24; A61L 2/10; A61L 2/18; A61L 2/26; G01H 3/00

(52) U.S. Cl. CPC A61L 2/24; A61L 2/10; A61L 2/18; A61L 2/26; G01H 3/00; A61L 2202/11; A61L 2202/14

(57) ABSTRACT

An aerosol reachable area estimation system includes a detector that detects a voice and a controller that estimates an area reachable by aerosol released to a space where an utterer who has emitted the voice detected by the detector exists from a speech sound included in the voice on a basis of a correlation between the speech sound and a velocity vector of aerosol released from the utterer when the utterer utters the speech sound and a position and a direction of the utterer's mouth.

### BACKGROUND

### 1. Technical Field

The present disclosure relates to an aerosol reachable area estimation system, an aerosol reachable area estimation method, and a non-transitory computer-readable recording medium used to estimate an area reachable by aerosol.

### 2. Description of the Related Art

Japanese Unexamined Patent Application Publication No. 2011-174624 discloses a method for detecting occurrence of coughing in a room and a position at which the coughing has occurred on the basis of a sound in the room. Japanese Unexamined Patent Application Publication No. 2020-030010 discloses a ventilation apparatus that detects occurrence of coughing or sneezing on the basis of a sound and that performs ventilation after a certain period of wait time.

S. Asadi et al., “Aerosol emission and superemission during human speech increase with voice loudness”, Nature Scientific Reports, February 2019, Vol. 9, No. 1 describes that there is a correlation between loudness of a voice and an area contaminated by aerosol caused by the voice. Philip Anfinrud et al., “Visualizing Speech-Generated Oral Fluid Droplets with Laser Light Scattering”, N Engl J Med 2020; 382:2061-2063 describes that in a case where a subject says, “Stay healthy”, an area contaminated by aerosol caused when “th” is pronounced is large.

### SUMMARY

With the above examples of the related art, however, it is difficult to accurately estimate an area reachable by aerosol caused by an utterer when the utterer has uttered speech sounds.

One non-limiting and exemplary embodiment provides an aerosol reachable area estimation system, an aerosol reachable area estimation method, and a non-transitory computer-readable recording medium capable of accurately estimating an area reachable by aerosol caused by an utterer when the utterer has uttered speech sounds.

In one general aspect, the techniques disclosed here feature an aerosol reachable area estimation system including a detector that detects a voice and a controller that estimates an area reachable by aerosol released to a space where an utterer who has emitted the voice detected by the detector exists from a speech sound included in the voice on a basis of a correlation between the speech sound and a velocity vector of aerosol released from the utterer when the utterer utters the speech sound and a position and a direction of the utterer's mouth.

With the aerosol reachable area estimation system according to the aspect of the present disclosure and the like, an area reachable by aerosol caused by an utterer when the utterer has uttered speech sounds can be accurately estimated.

It should be noted that these general or specific aspects may be implemented as an apparatus, a system, a method, an integrated circuit, a computer program, a computer-readable storage medium, or any selective combination thereof. The computer-readable storage medium includes, for example, a nonvolatile storage medium such as a compact disc read-only memory (CD-ROM).

Additional benefits and advantages of the disclosed embodiments will become apparent from the specification and drawings. The benefits and/or advantages may be individually obtained by the various embodiments and features of the specification and drawings, which need not all be provided in order to obtain one or more of such benefits and/or advantages.

### BRIEF DESCRIPTION OF THE DRAWINGS

FIG. 1 illustrates a schlieren image of a side of a person's mouth at a time when the person utters a monosyllable “a” (Japanese);

FIG. 2 illustrates a schlieren image of a side of a person's mouth at a time when the person utters a monosyllable “i” (Japanese);

FIG. 3 is a diagram illustrating a method for measuring a velocity vector of aerosol caused by speech using a schlieren image of a side of a person's mouth at a time when the person utters a monosyllable “hi” (Japanese);

FIG. 4 is a block diagram illustrating an example of the configuration of a contamination area estimation system according to an embodiment;

FIG. 5 is a diagram illustrating a result of measurement of velocity vectors at times when monosyllables of vowels in Japanese are uttered;

FIG. 6 is a diagram illustrating a part of a result of measurement of velocity vectors at times when monosyllables including consonants in Japanese are uttered;

FIG. 7 is a diagram illustrating relationships between phonetic symbols, places of articulation, and widths of the mouth (how wide the mouth opens) of vowels established by the International Phonetic Association;

FIG. 8 is a diagram illustrating relationships between phonetic symbols, manners of articulation, and places of articulation of monosyllables including consonants similarly established by the International Phonetic Association;

FIG. 9 is a diagram illustrating a method for estimating a contamination area at a time when an utterer in a space is viewed from a side;

FIG. 10 is a diagram illustrating a method for estimating a contamination area at a time when the utterer in the space is viewed from above;

FIG. 11 is a flowchart illustrating an example of an operation performed by the contamination area estimation system according to the embodiment;

FIG. 12 is a block diagram illustrating an example of the configuration of a contamination area estimation system according to a first modification of the embodiment;

FIG. 13 is a diagram illustrating a result of measurement of sound pressure dependence of the number of aerosol particles released when a monosyllable “shi” (Japanese) is uttered once;

FIG. 14 is a flowchart illustrating an example of an operation performed by the contamination area estimation system according to the first modification of the embodiment;

FIG. 15 is a diagram illustrating a specific example of a process performed by an aerosol quantity analysis portion; and

FIG. 16 is a block diagram illustrating an example of the configuration of a contamination area estimation system according to a second modification of the embodiment.

### DETAILED DESCRIPTIONS

### Underlying Knowledge Forming Basis of Present Disclosure

Aerosol released from a person's mouth when he/she sneezes, coughs, or speaks generally includes droplets. Droplets tend to fall from a person's mouth. The aerosol also includes droplet nuclei of approximately 10 μm or less in size formed through evaporation of moisture contained in the droplets. The droplet nuclei tend to remain in air without falling.

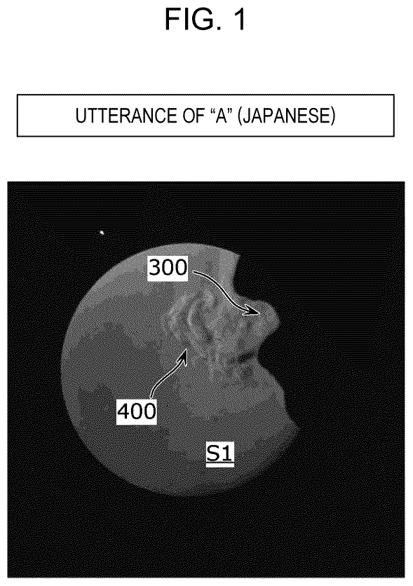

FIG. 1 illustrates a schlieren image of a side of a person's mouth at a time when the person utters a monosyllable “a” (Japanese). A schlieren image refers to an image captured through schlieren optical measurement, which is herein used as a method for visualizing aerosol released through speech using a difference in a refractive index derived from a density difference between the aerosol and surrounding air. As illustrated in FIG. 1, when a person utters a monosyllable “a” (Japanese), aerosol 400 released to a space S1 from the person's mouth 300 spreads almost straight out horizontally.

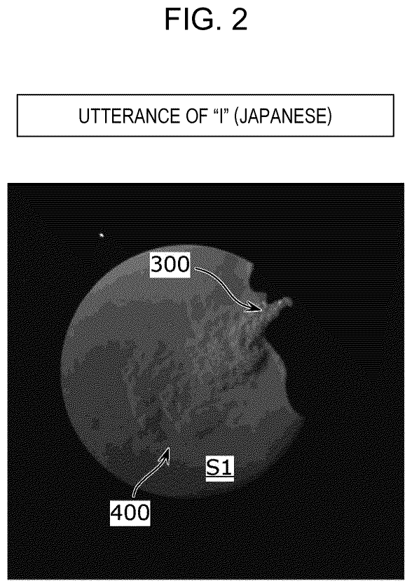

FIG. 2 illustrates a schlieren image of a side of a person's mouth at a time when the person utters a monosyllable “i” (Japanese). Compared to FIG. 1, where a person utters “a” (Japanese), aerosol released to the space S1 from the person's mouth 300 when the person utters “i” (Japanese) travels diagonally downward from the person's mouth 300. An area over which aerosol spreads thus differs depending on a speech sound uttered by a person.

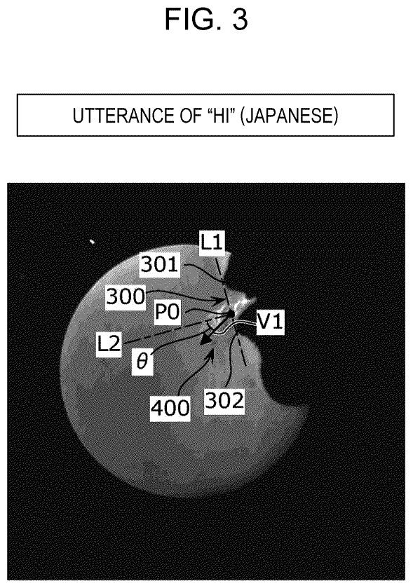

FIG. 3 is a diagram illustrating a method for measuring a velocity vector of aerosol caused by speech using a schlieren image of a side of a person's mouth at a time when the person utters a monosyllable “hi” (Japanese). As illustrated in FIG. 3, a velocity vector V1 of aerosol 400 is calculated, by obtaining, with a position P0 on a tangent line L1 to an upper lip 301 and a lower lip 302 set as an origin, a travel distance and an angle of the aerosol 400 10 ms after the aerosol 400 is released from the person's mouth 300. The velocity vector V1 includes an initial velocity, which is obtained by dividing the travel distance by 10 ms, and an obtained angle θ. The angle θ is an angle to a line L2 perpendicular to the tangent line L1, and takes a positive value above the line L2 and a negative value below the line L2. The velocity vector V1 is associated with a phonetic symbol of each speech sound (monosyllable). The speech sounds herein refer to sounds used as language and sounds other than a cough, a sneeze, and other non-speech sounds. The monosyllables herein refer to speech sounds broken down to basic sounds (e.g., “hi”), dakuten sounds (e.g., “bi”), handakuten sounds (e.g., “pi”), or the like. That is, each speech sound includes one or more monosyllables. Vowels are “a”, “i”, “u”, “e”, and “o” among the basic sounds.

Although Japanese Unexamined Patent Application Publication No. 2011-174624 and Japanese Unexamined Patent Application Publication No. 2020-030010 take into consideration a risk of transmission of infectious disease due to a cough or a sneeze, these examples of the related art do not take into consideration the fact that, when an utterer utters a speech sound, a velocity vector of aerosol released from the utterer's mouth differs depending on the speech sound as described with reference to FIGS. 1 to 3.

Neither S. Asadi et al., “Aerosol emission and superemission during human speech increase with voice loudness”, Nature Scientific Reports, February 2019, Vol. 9, No. 1 nor Philip Anfinrud et al., “Visualizing Speech-Generated Oral Fluid Droplets with Laser Light Scattering”, N Engl J Med 2020; 382:2061-2063 describe the fact that a velocity vector of aerosol released from a person's mouth differs depending on the speech sound.

The above examples of the related art thus have a problem that it is difficult to accurately estimate an area reachable by aerosol released when an utterer has uttered speech sounds, that is, an area that can be contaminated by the aerosol. It is therefore undesirably difficult to reduce the risk of infection sufficiently.

In order to solve the above problem, an aerosol reachable area estimation system according to an aspect of the present disclosure includes a detector that detects a voice and a controller that estimates an area reachable by aerosol released to a space where an utterer who has emitted the voice detected by the detector exists from a speech sound included in the voice on a basis of a correlation between the speech sound and a velocity vector of aerosol released from the utterer when the utterer utters the speech sound and a position and a direction of the utterer's mouth.

As a result, an area reachable by aerosol released from an utterer to a space where the utterer exists when the utterer has uttered speech sounds can be accurately estimated.

The correlation may be a correlation between way the speech sound is uttered and the velocity vector. The controller may (i) identify the way the speech sound included in the voice is uttered and (ii) estimate the area on a basis of the velocity vector associated in the correlation with the identified way the speech sound is uttered and the position and the direction of the utterer's mouth.

As a result, a velocity vector can be identified on the basis of a correlation between a way a speech sound is uttered and a velocity vector, thereby reducing the amount of data regarding the correlation.

The way the speech sound is uttered may differ depending on a place of articulation used to utter the speech sound.

As a result, a velocity vector can be identified on the basis of a correlation between a place of articulation and a velocity vector, thereby reducing the amount of data regarding the correlation.

The way the speech sound is uttered may differ depending on a manner of articulation used to utter the speech sound.

As a result, a velocity vector can be identified on the basis of a correlation between a manner of articulation and a velocity vector, thereby reducing the amount of data regarding the correlation.

The correlation may indicate that magnitude of the velocity vector becomes greater as the utterer's mouth opens smaller when the utterer utters the speech sound.

In the correlation, a direction of a velocity vector associated with a speech sound uttered with a place of articulation such as teeth, an alveolar ridge, or a back of the alveolar ridge may be more downward than a direction of a velocity vector associated with a speech sound uttered with another place of articulation.

The aerosol reachable area estimation system may further include a mouth detector that detects the position and the direction of the utterer's mouth. The controller may obtain the position and the direction on a basis of a result of the detection performed by the mouth detector.

As a result, a position and a direction of an utterer's mouth can be accurately identified, and an area reachable by aerosol can be accurately estimated.

The mouth detector may be a camera that captures an image of the utterer's mouth. The controller may analyze the image captured by the camera and identify the position and the direction.

As a result, a position and a direction of an utterer's mouth can be accurately identified using an image captured by the camera, and an area reachable by aerosol can be accurately estimated.

The aerosol reachable area estimation system may further include an obtainer that obtains object information indicating arrangement of an object in the space where the utterer exists. The controller may identify the position and the direction on a basis of the object information.

As a result, a position of an utterer's mouth can be estimated on the basis of object information.

The aerosol reachable area estimation system may further include an infrared sensor that detects presence or absence of a person. The controller may identify the position and the direction on a basis of the object information and a result of the detection performed by the infrared sensor.

As a result, presence of an utterer can be detected in a detection area of the infrared sensor. The controller, therefore, can accurately detect a position of the utterer's mouth.

The aerosol reachable area estimation system may further include an ultrasonic sensor that detects the space. The controller may (i) identify a position of an object in the space on a basis of a result of the detection performed by the ultrasonic sensor and (ii) identify the position and the direction on a basis of the identified position of the object.

As a result, a position or a size of an object in a space can be identified on the basis of a result of detection performed by the ultrasonic sensor.

The controller may notify of the estimated area.

As a result, an area reachable by aerosol can be notified of.

The controller may notify of the estimated area using an apparatus outside the aerosol reachable area estimation system.

As a result, a notification can be issued to an utterer or a user in a space.

The apparatus outside the aerosol reachable area estimation system may be a display apparatus provided in the space or a mobile terminal owned by the utterer.

As a result, a notification can be issued to an utterer or a user in a space.

The controller may spray a disinfectant solution or radiate ultraviolet light onto the estimated area using the apparatus outside the aerosol reachable area estimation system.

As a result, a disinfectant solution can be sprayed or ultraviolet light can be radiated onto an area reachable by aerosol. The disinfectant solution, therefore, can be sprayed or ultraviolet light can be radiated effectively, thereby reducing a risk of infection.

The controller may ventilate the space including the estimated area.

As a result, a space including an area reachable by aerosol can be ventilated, thereby reducing the risk of infection. The ventilation may be performed by a ventilation fan, an air purifier, an air circulator fan, or the like.

An aerosol reachable area estimation method according to another aspect of the present disclosure includes detecting a voice and estimating an area reachable by aerosol released to a space where an utterer who has emitted the voice detected in the detecting exists from a speech sound included in the voice on a basis of a correlation between the speech sound and a velocity vector of aerosol released from the utterer when the utterer utters the speech sound and a position and a direction of the utterer's mouth.

As a result, an area reachable by aerosol released from an utterer to a space where the utterer exists when the utterer has uttered speech sounds can be accurately estimated.

It should be noted that these general or specific aspects may be implemented as an apparatus, an integrated circuit, a computer program, a computer-readable storage medium such as a CD-ROM, or any selective combination thereof.

Embodiments will be specifically described hereinafter with reference to the drawings. An aerosol reachable area estimation system will be described as a contamination area estimation system, and an aerosol reachable area estimation method will be described as a contamination area estimation method.

The embodiments described hereinafter are general or specific examples. Values, shapes, materials, components, arrangement positions and connection modes of the components, steps, order of the steps are examples, and are not intended to limit the present disclosure. Among the components mentioned in the following embodiments, ones not described in the independent claims will be described as optional components.

The drawings are schematic diagrams, and not necessarily strict illustrations. Scales and the like, therefore, do not necessarily match between the drawings. In the drawings, essentially the same components are given the same reference numerals, and redundant description thereof is omitted or simplified.

Ranges of values herein are not strict, and include essentially the same ranges with a difference of, say, several percent.

### Embodiment

### 1-1. Configuration

First, the configuration of the contamination area estimation system according to an embodiment will be described.

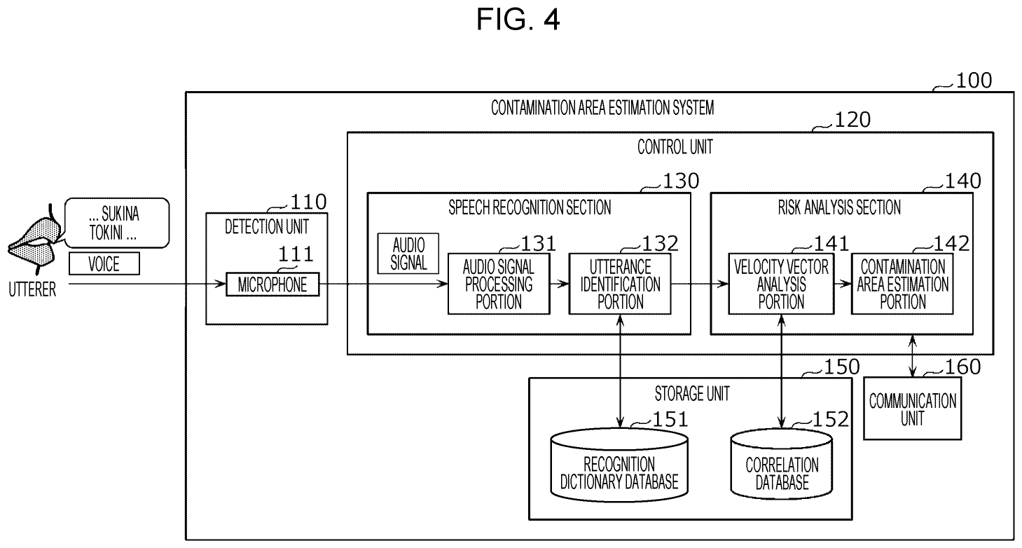

FIG. 4 is a block diagram illustrating an example of the configuration of the contamination area estimation system according to the first embodiment.

A contamination area estimation system 100 includes a detection unit 110, a control unit 120, a storage unit 150, and a communication unit 160.

The detection unit 110 includes a microphone 111 that detects a voice. The detection unit 110 detects, for example, a voice emitted by an utterer. The microphone 111 detects a voice emitted by an utterer and converts the detected voice into an audio signal. The audio signal obtained as a result of the conversion is output to the control unit 120.

The control unit 120 estimates, from speech sounds included in a voice detected by the detection unit 110 on the basis of first correlations (described later) stored in the storage unit 150 in advance and a position and a direction of an utterer's face, a contamination area, which is contaminated by aerosol released to a space where the utterer who has emitted the voice exists. The control unit 120 includes a speech recognition section 130 and a risk analysis section 140.

The speech recognition section 130 identifies, on the basis of an audio signal, each of monosyllables included in a voice indicated by the audio signal. The speech recognition section 130 includes an audio signal processing portion 131 and an utterance identification portion 132.

The audio signal processing portion 131 performs a sound pressure determination, noise reduction, and signal processing (sound analysis) such as a spectrum transformation on an audio signal. The audio signal processing portion 131 need not make the sound pressure determination in the present embodiment. The audio signal processing portion 131 calculates mel-frequency cepstral coefficients (MFCCs), which are feature values of speech sounds, from an audio signal as acoustic feature values. The MFCCs are feature values indicating vocal tract characteristics of an utterer and generally used in speech recognition. More specifically, the MFCCs are acoustic feature values obtained by analyzing frequency spectra of speech sounds on the basis of auditory characteristics of humans. The audio signal processing portion 131 may calculate an audio signal subjected to a mel-filterbank or a spectrogram of an audio signal as acoustic feature values. The audio signal processing portion 131 calculates acoustic feature values for each of monosyllables included in a voice. The audio signal processing portion 131 outputs one or more acoustic feature values calculated on the basis of an audio signal to the utterance identification portion 132.

The utterance identification portion 132 performs, using a recognition dictionary database 151 stored in the storage unit 150, utterance identification on acoustic feature values (an audio signal subjected to signal processing). The utterance identification portion 132 performs utterance identification based on machine learning. That is, the utterance identification portion 132 identifies each of one or more monosyllables included in speech sounds included in a voice indicated by an audio signal. A result of the utterance identification is output to the risk analysis section 140.

The recognition dictionary database 151 is, for example, a dictionary for recognizing monosyllables in speech sounds and includes a large number of pairs of acoustic feature values and correct data regarding a monosyllable in a speech sound. The utterance identification portion 132 is a machine learning model. Before performing the utterance identification (i.e., recognizing monosyllables in speech sounds), the utterance identification portion 132 is tuned using the recognition dictionary database 151 as training data. The utterance identification portion 132 includes an input layer, hidden layers, and an output layer.

Inputs of the utterance identification portion 132 are acoustic feature values.

Outputs of the utterance identification portion 132 are information for identifying speech sounds (monosyllables), and units included in the output layer of the utterance identification portion 132 output the information for identifying speech sounds (monosyllables). The information for identifying speech sounds (monosyllables) and the units included in the output layer of the utterance identification portion 132 are in one-to-one correspondence.

In the utterance identification, a first acoustic feature value included in one or more acoustic feature values is input to the utterance identification portion 132 and the utterance identification portion 132 outputs information for identifying a first speech sound (monosyllable), . . . , and an n-th acoustic feature value included in the one or more acoustic feature values is input to the utterance identification portion 132 and the utterance identification portion 132 outputs information for identifying an n-th speech sound (monosyllable). n is an integer larger than or equal to 1 and is equal to the number of one or more acoustic feature values.

The utterance identification portion 132 outputs, to the risk analysis section 140, information for identifying one or more speech sounds (monosyllables) corresponding to the one or more acoustic feature values. That is, the utterance identification portion 132 outputs, to the risk analysis section 140, the information for identifying the first speech sound (monosyllable) corresponding to the first acoustic feature value, . . . , and the information for identifying the n-th speech sound (monosyllable) corresponding to the n-th acoustic feature value.

The recognition dictionary database 151 need not necessarily include a dictionary for recognizing all monosyllables in speech sounds, and may include only a dictionary for recognizing one or more monosyllables in certain speech sounds. In this case, the utterance identification portion 132 may identify monosyllables uttered by an utterer only for certain speech sounds. The number of units included in the output layer of the utterance identification portion 132 may be the number of one or more monosyllables in the certain speech sounds.

The risk analysis section 140 estimates an area contaminated by each monosyllable when an utterer has uttered the monosyllable from a corresponding result of utterance identification obtained from the speech recognition section 130 (e.g., the information for identifying the first speech sound (monosyllable), . . . , and the information for identifying the n-th speech sound (monosyllable)). The risk analysis section 140 includes a velocity vector analysis portion 141 and a contamination area estimation portion 142.

The velocity vector analysis portion 141 estimates a velocity vector of each of monosyllables included in a result of utterance identification using the correlation database 152 stored in the storage unit 150. The velocity vector analysis portion 141 estimates, on the basis of the first correlations, velocity vectors from speech sounds included in a voice detected by the detection unit 110. For example, the velocity vector analysis portion 141 estimates a velocity vector of aerosol caused when an utterer has uttered each monosyllable included in a result of utterance identification performed by the utterance identification portion 132 by reading a first correlation corresponding to the monosyllable from the correlation database 152 and identifying a velocity vector corresponding to the monosyllable in the read first correlation. The velocity vector analysis portion 141 estimates a velocity vector of each monosyllable.

The correlation database 152 will be described hereinafter.

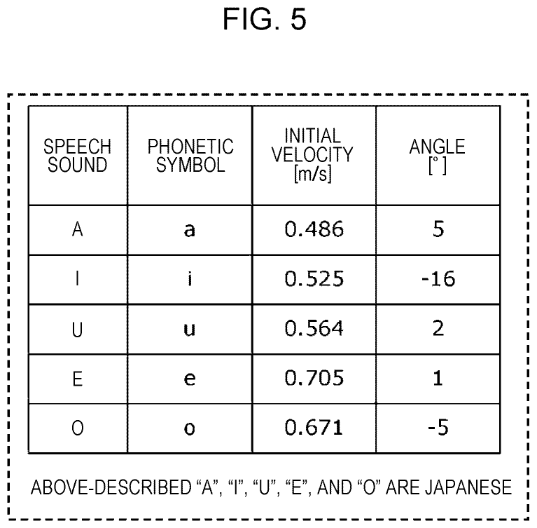

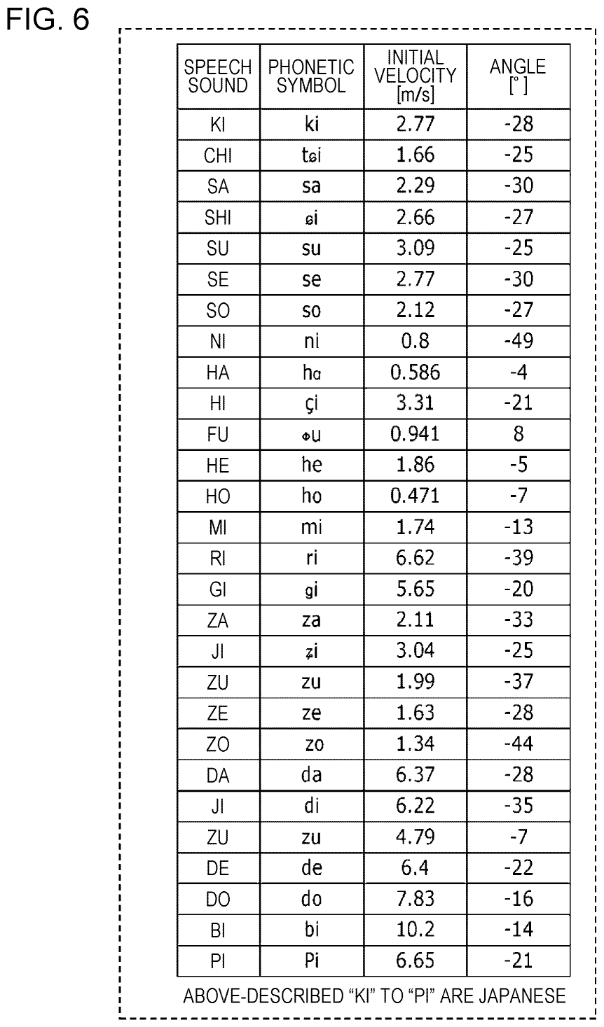

The correlation database 152 includes first correlations indicating relationships between certain speech sounds and velocity vectors of aerosol released from an utterer when the utterer utters the certain speech sounds. The first correlations indicate, as in FIGS. 5 and 6, for example, velocity vectors of aerosol released when monosyllables in speech sounds are uttered.

FIG. 5 is a diagram illustrating a result of measurement of velocity vectors at times when monosyllables of vowels in Japanese are uttered. FIG. 6 is a diagram illustrating a part of a result of measurement of velocity vectors at a time when monosyllables including consonants in Japanese are uttered. As illustrated in FIGS. 5 and 6, a velocity vector when each speech sound (monosyllable) is uttered is measured for the speech sound, and the speech sound and the velocity vector when the speech sound is uttered are associated with each other. First correlations where velocity vectors V1 and phonetic symbols are associated with each other may be stored in a storage unit as a correlation database.

As a result, when a voice is detected, a velocity vector of aerosol released by the voice can be estimated by referring to velocity vectors associated in the correlation database with speech sounds included in the detected voice. Because monosyllables are successively uttered in actual conversations (utterances), complex behavior is expected. In this case, monosyllables with especially high velocities or especially loud monosyllables may be extracted from among successively uttered monosyllables, and velocity vectors of the extracted monosyllables may be estimated and then combined.

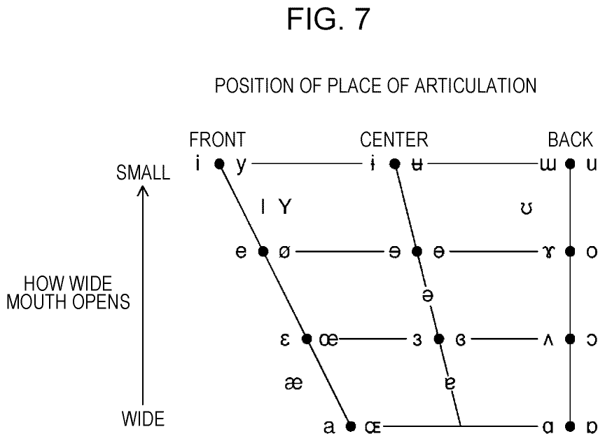

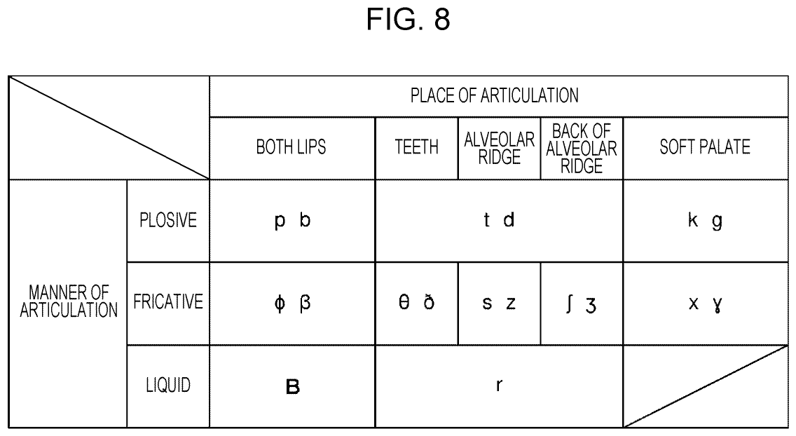

FIG. 7 is a diagram illustrating relationships between phonetic symbols, places of articulation, and widths of the mouth (how wide the mouth opens) of vowels established by the International Phonetic Association. FIG. 8 is a diagram illustrating relationships between phonetic symbols, manners of articulation, and places of articulation of monosyllables including consonants similarly established by the International Phonetic Association. FIG. 8 is a diagram illustrating relationships between phonetic symbols, manners of articulation, and places of articulation of monosyllables with which magnitude of velocity vectors when aerosol is released (i.e., initial velocities) tends to be greater than a certain velocity.

Characteristics of the velocity vectors of the monosyllables in Japanese illustrated in FIGS. 5 and 6 indicate the following. That is, monosyllables uttered with places of articulation around an alveolar ridge tend to be associated with negative (downward) angles of release of aerosol. That is, in the first correlations, angles (directions) of velocity vectors associated with speech sounds uttered with places of articulation around the alveolar ridge are more downward than angles (directions) of velocity vectors associated with speech sounds uttered with other places of articulation.

Monosyllables uttered with places of articulation other than around the alveolar ridge tend to be associated with angles of release of aerosol close to ones associated with vowels included therein. The angles close to those associated with the vowels refer to, for example, angles whose differences from the angles associated with the vowels (i.e., “a” (Japanese), “i” (Japanese), “u” (Japanese), “e” (Japanese), and “o” (Japanese)) are smaller than a certain value. Angles (directions) at which aerosol is released from an utterer when the utterer utters monosyllables in an “a” row (Japanese) and “u” row (Japanese), for example, tend to be ones with high straightness from the utterer's mouth in a horizontal direction. Angles (directions) at which aerosol is released from an utterer when the utterer utters monosyllables in an “i” row (Japanese) tend to be ones more downward than the horizontal direction from the utterer's mouth. The places of articulation around the alveolar ridge include teeth, the alveolar ridge, and a back of the alveolar ridge.

Monosyllables whose initial velocities of released aerosol are higher than a certain velocity (e.g., 3 m/s) tend to satisfy one of conditions including (i) a place of articulation is around the alveolar ridge, (ii) a manner of articulation is a plosive, a fricative, or a liquid, and (iii) the mouth opens small. More specifically, when the mouth opens small, the mouth opens as when the phonetic symbols of the vowels in top two of four stages illustrated in FIG. 7 are uttered. The first correlations indicate that the magnitude of a velocity vector becomes greater as the utterer's mouth opens smaller when the utterer utters a speech sound.

Because velocity vectors of monosyllables tend to be similar to each other depending on ways the monosyllables are uttered as illustrated in FIGS. 7 and 8, the monosyllables can be classified into different groups in accordance with the ways. That is, in the first correlations, a velocity vector need not be associated with each monosyllable and may be associated with each of groups of monosyllables based on the ways monosyllables belonging to the group are uttered. That is, the first correlations may be ones between ways speech sounds are uttered and velocity vectors.

The groups based on the ways speech sounds are uttered may be ones based on places of articulation used to utter the speech sounds. Velocity vectors, therefore, can be identified on the basis of the correlations between the places of articulation and the velocity vectors, thereby reducing the amount of data regarding the correlations.

The groups based on the ways speech sounds are uttered may be ones based on manners of articulation used to utter the speech sounds. Velocity vectors, therefore, can be identified on the basis of the correlations between the manners of articulation and the velocity vectors, thereby reducing the amount of data regarding the correlations.

The correlation database 152 may thus include first correlations between monosyllables in speech sounds uttered by an utterer and velocity vectors of aerosol released from the utterer. The correlation database 152 may include first correlations between groups based on ways speech sounds are uttered to which monosyllables in speech sounds uttered by an utterer belong and velocity vectors of aerosol released from the utterer. In this case, the velocity vector analysis portion 141 may identify ways speech sounds included in a voice are uttered and then identify, in the first correlations, velocity vectors corresponding to the ways.

The correlation database 152 need not necessarily include first correlations regarding all monosyllables in speech sounds, and may include only first correlations regarding one or more monosyllables in certain speech sounds. In this case, the velocity vector analysis portion 141 may estimate velocity vectors of monosyllables uttered by the utterer only for certain speech sounds.

The certain speech sounds may be monosyllables whose initial velocities of released aerosol are higher than the certain velocity (e.g., 3 m/s).

Next, the contamination area estimation portion 142 will be described.

The contamination area estimation portion 142 estimates, for each speech sound (monosyllable) included in a voice emitted by an utterer, a contamination area, which is contaminated by aerosol released from the utterer when the utterer has uttered the monosyllable, on the basis of a velocity vector estimated by the velocity vector analysis portion 141 for the monosyllable and a position and a direction of the utterer's face.

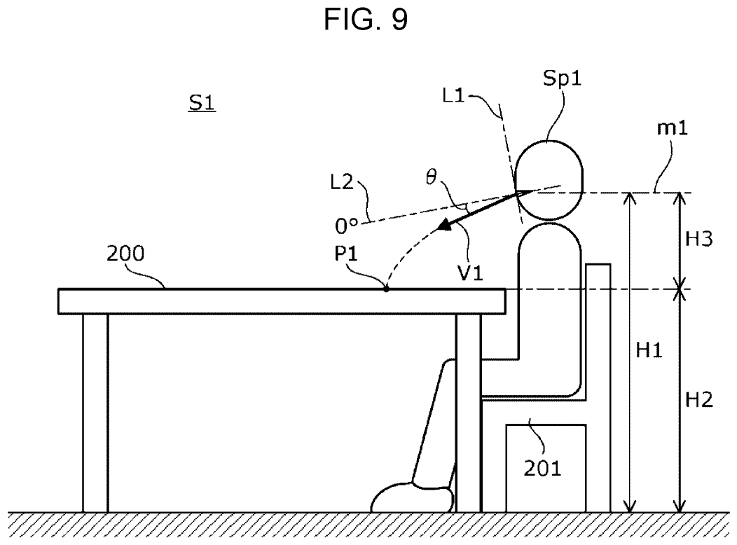

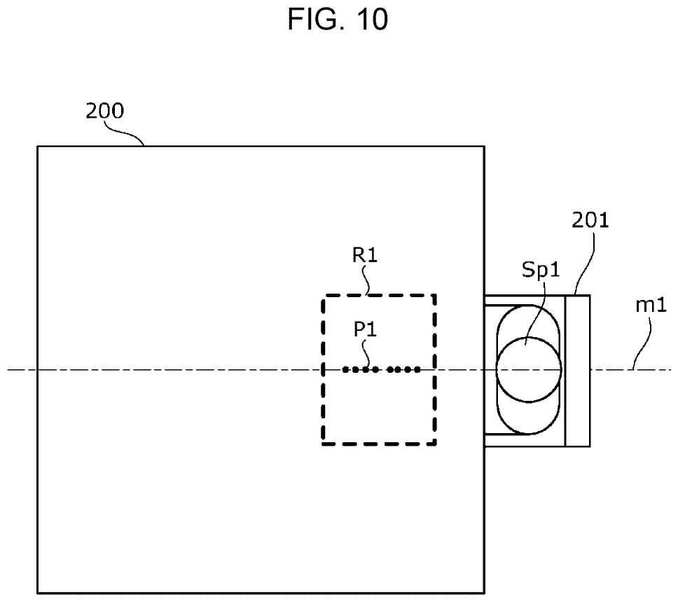

FIG. 9 is a diagram illustrating a method for estimating a contamination area at a time when an utterer in a space is viewed from a side. FIG. 10 is a diagram illustrating a method for estimating a contamination area at a time when the utterer in the space is viewed from above.

FIGS. 9 and 10 illustrate a situation where an utterer Sp1 is sitting in a space S1 on a chair 201 provided beside a table 200. It is estimated that when the utterer Sp1 sitting on the chair 201 utters a monosyllable, aerosol with a velocity vector V1 based on the uttered monosyllable is released from the utterer Sp1. At this time, if a position and a direction of a face of the utterer Sp1 can be identified, an area over which aerosol is released can be estimated since the aerosol is estimated to be released from a mouth of the utterer Sp1 at the velocity vector V1.

The contamination area estimation portion 142 obtains, from the storage unit 150, the position and the direction of the face of the utterer Sp1 at a time when the utterer Sp1 is sitting on the chair 201 and estimates, as a contamination area, an area over which aerosol is released when the aerosol is released from the mouth of the utterer Sp1 at the velocity vector V1 in the direction of the face of the utterer Sp1, with a position of the mouth of the utterer Sp1 obtained from the position and the direction of the face of the utterer Sp1 set as an origin. More specifically, the contamination area estimation portion 142 may estimate a position P1 on a top of the table 200 at which released aerosol (droplets) reaches. When it is estimated that the utterer Sp1 is likely to be in the space S1 such as when the table 200 and the chair 201 are arranged at fixed positions in the space S1, for example, the storage unit 150 may store a position and a direction of the face of the utterer Sp1 set in advance.

The storage unit 150 need not store the position and the direction of the face of the utterer Sp1 set in advance and may store object information indicating arrangement of objects in the space S1. In this case, the contamination area estimation portion 142 may identify the position and the direction of the face of the utterer Sp1 on the basis of the object information. The object information may include, for example, a position of the table 200 in the space S1, size of the table 200, a position of the chair 201 in the space S1, and size of the chair 201. Since an area where an utterer is located can be identified on the basis of the object information, a position of the utterer's face can be estimated.

The contamination area estimation portion 142 estimates the position and the direction of the face of the utterer Sp1 sitting on the chair 201. The contamination area estimation portion 142 may estimate the position P1 on the top of the table 200 at which released aerosol (droplets) reaches by calculating a height H3 of the face from the top of the table 200, the height H3 being obtained by subtracting a height H2 of the table 200 from a height H1 of a position ml of the mouth when the utterer Sp1 is sitting on the chair 201, and a distance in the horizontal direction over which aerosol released at the velocity vector V1 travels before dropping by the height H3.

As illustrated in FIG. 10, the contamination area estimation portion 142 may estimate, for different monosyllables, positions P1 on the top of the table 200 at which droplets reaches and a contamination area R1 including the estimated positions P1 corresponding to the monosyllables as a contamination area. The contamination area R1 is an area encompassing the positions P1 and is a rectangular area defined by a width including the positions P1 in a direction ml of the utterer Sp1 (i.e., a front-and-back direction of the utterer Sp1) and a certain width in a direction perpendicular to the direction ml when viewed from above. The certain width is a range over which aerosol is estimated to extend in the direction perpendicular to the direction ml and may be a predetermined fixed range. The contamination area R1 is not limited to a rectangular area insofar as the positions P1 are encompassed, and may be a circular area, an elliptical area, a fan-shaped area that extends from the utterer Sp1.

The contamination area estimation portion 142 sequentially estimates positions on the top of the table 200 at which estimated aerosol reaches for monosyllables uttered by the utterer Sp1 and sequentially updates the contamination area R1. The contamination area estimation portion 142 may determine whether area of the estimated contamination area R1 exceeds a certain area and, if the area of the contamination area R1 exceeds the certain area, determine that the risk of transmission of infectious disease is high. The certain area is stored, for example, in the storage unit 150. If the area of the calculated contamination area R1 exceeds the certain area, the contamination area estimation portion 142 may issue a notification. The contamination area estimation portion 142 need not determine whether the area of the contamination area R1 exceeds the certain area, and may issue a notification each time the contamination area estimation portion 142 estimates the contamination area R1. For example, the contamination area estimation portion 142 may issue a notification by controlling the communication unit 160 in such a way as to transmit notification information indicating the notification to an external apparatus outside the contamination area estimation system 100. The external apparatus to which the notification information is transmitted may be, for example, a display apparatus provided in a space where the microphone 111 of the contamination area estimation system 100 is provided or the mobile terminal owned by the utterer. Addresses of the display apparatus and the mobile terminal in this case may be registered to the contamination area estimation system 100 in advance. The display apparatus may be included in the contamination area estimation system 100 and display a result of processing performed by the contamination area estimation portion 142.

If determining that the risk of infection is high, the contamination area estimation portion 142 may notify the utterer of a result of the determination (e.g., a warning indicating that the risk of infection is high). A warning can thus be issued if the risk of infection is high, and the user can be prompted to take measures to reduce a contamination area.

The contamination area estimation portion 142 may determine the risk of infection itself, instead of determining whether the risk of infection is high. For example, the contamination area estimation portion 142 may determine a higher risk of infection as the area of the contamination area R1 increases. The result of the determination may be classifications of different levels such as “there is some risk of infection”, “risk of infection is high”, and “risk of infection is very high”, or may be a value indicating the risk of infection.

The storage unit 150 stores the recognition dictionary database 151 and the correlation database 152.

The communication unit 160 is communicably connected to a communication network. The communication unit 160 may communicate with an external apparatus (e.g., a server) outside the contamination area estimation system 100 over the communication network. The communication unit 160 may communicate with the server, for example, and receive new first correlations between speech sounds and velocity vectors from the server. The new first correlations may be, for example, more accurate first correlations generated by the server using more results of experiments or the like. When the communication unit 160 receives new first correlations, the control unit 120 controls the communication unit 160 such that the first correlations stored in the correlation database 152 of the storage unit 150 are updated to the new first correlations received by the communication unit 160. The communication unit 160 need not necessarily receive new first correlations from the server, and may receive a new dictionary for improving accuracy of identifying utterances or a certain new area (threshold) for issuing a notification. In this case, when the communication unit 160 receives the new dictionary from the server, the control unit 120 may control the communication unit 160 such that the dictionary stored in the recognition dictionary database 151 of the storage unit 150 is updated to the new dictionary received by the communication unit 160. When the communication unit 160 receives the new certain area (threshold) from the server, the control unit 120 may control the communication unit 160 such that the certain area (threshold) stored in the storage unit 150 is updated to the certain area (threshold).

The communication unit 160 may obtain infection information indicating a relationship between a contamination area and a risk of viral infection or a relationship between a contamination area and a state of infection from the server. The infection information may be used in the determination of the risk of infection made by the contamination area estimation portion 142.

### 1-2. Operation

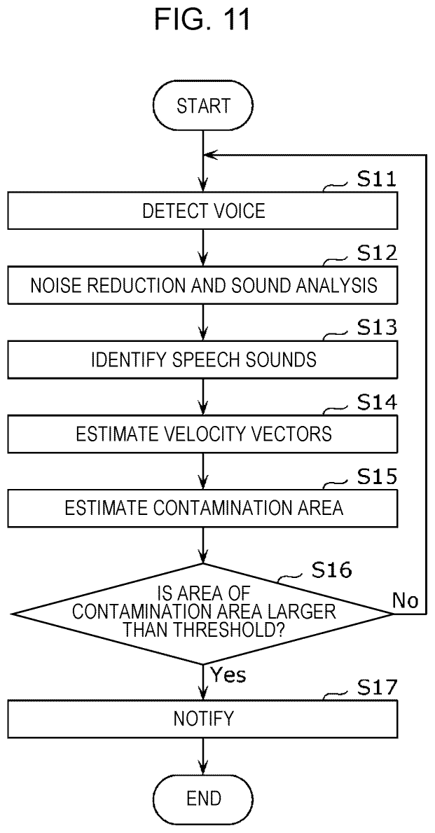

Next, an operation performed by the contamination area estimation system 100 according to the present embodiment will be described with reference to FIG. 11. FIG. 11 is a flowchart illustrating an example of the operation performed by the contamination area estimation system according to the present embodiment.

First, in the contamination area estimation system 100, the detection unit 110 detects a voice with the microphone 111 and generates an audio signal (S11).

Next, the audio signal processing portion 131 of the speech recognition section 130 in the control unit 120 reduces noise in the generated audio signal and performs signal processing (sound analysis) such as a spectrum transformation (S12).

Next, the utterance identification portion 132 of the speech recognition section 130 performs, using the recognition dictionary database 151, utterance identification on the audio signal subjected to the signal processing. As a result, the utterance identification portion 132 identifies speech sounds, or groups based on ways speech sounds are uttered, to which monosyllables included in speech sounds included in the voice indicated by the audio signal are classified (S13). The utterance identification portion 132 may identify certain speech sounds among the monosyllables included in the speech sounds included in the voice indicated by the audio signal.

Next, the velocity vector analysis portion 141 of the risk analysis section 140 estimates a velocity vector for each monosyllable included in a result of the utterance identification using the correlation database 152 (S14). The velocity vector analysis portion 141 reads a first correlation corresponding to each monosyllable included in the result of the utterance identification performed by the utterance identification portion 132 and identifies the velocity vector corresponding, in the read first correlation, to the monosyllable.

Next, the contamination area estimation portion 142 estimates, for each speech sound (monosyllable) included in the voice, a contamination area, which is contaminated by aerosol released from an utterer when the utterer has uttered the monosyllable, on the basis of the velocity vector estimated by the velocity vector analysis portion 141 for the monosyllable and a position and a direction of the utterer's face (S15). For example, the contamination area estimation portion 142 stores, in the storage unit 150, the position P1 on the top of the table 200 at which the aerosol estimated to calculate a contamination area R1 reaches. As a result, the contamination area estimation portion 142 can calculate a new contamination area R1 by adding the position P1 obtained through the estimation in step S15 to a position P1 calculated in a previous estimation. If the previous position P1 is not stored in the storage unit 150, the contamination area R1 is estimated on the basis of the position P1 obtained this time.

Next, the contamination area estimation portion 142 determines whether the area of the contamination area R1 is larger than a certain area (S16).

If the area of the contamination area R1 is larger than the certain area (YES in S16), the contamination area estimation portion 142 issues a notification (S17). If the area of the contamination area R1 is smaller than or equal to the certain area (NO in S16), the contamination area estimation portion 142 returns to step S11. That is, after estimating the contamination area R1 or if determining that a notification need not be issued, the contamination area estimation portion 142 performs the same process again. The contamination area estimation system 100 may keep performing the same process even after a notification is issued.

### 1-3. Advantageous Effects

As described above, the contamination area estimation system 100 according to the present embodiment includes the detection unit 110 that detects a voice and the control unit 120. The control unit 120 estimates a contamination area, which is contaminated by aerosol released to a space where an utterer who has emitted a voice detected by the detection unit 110 exists, from speech sounds included in the voice on the basis of correlations between the speech sounds and velocity vectors of aerosol released from the utterer when the utterer utters the speech sounds and a position and a direction of the utterer's face.

A contamination area, which is contaminated by aerosol released from an utterer to a space where the utterer exists when the utterer has uttered speech sounds, therefore, can be accurately estimated.

In the contamination area estimation system 100, the correlations are correlations between ways speech sounds are uttered and velocity vectors. The control unit 120 may (i) identify ways speech sounds included in a voice are uttered and (ii) estimate a contamination area on the basis of velocity vectors corresponding, in the correlations, to the identified ways and a position and a direction of a face. Velocity vectors can thus be identified on the basis of correlations between ways speech sounds are uttered and velocity vectors, thereby reducing the amount of data regarding the correlations.

### 1-4. First Modification

Next, a first modification of the embodiment will be described.

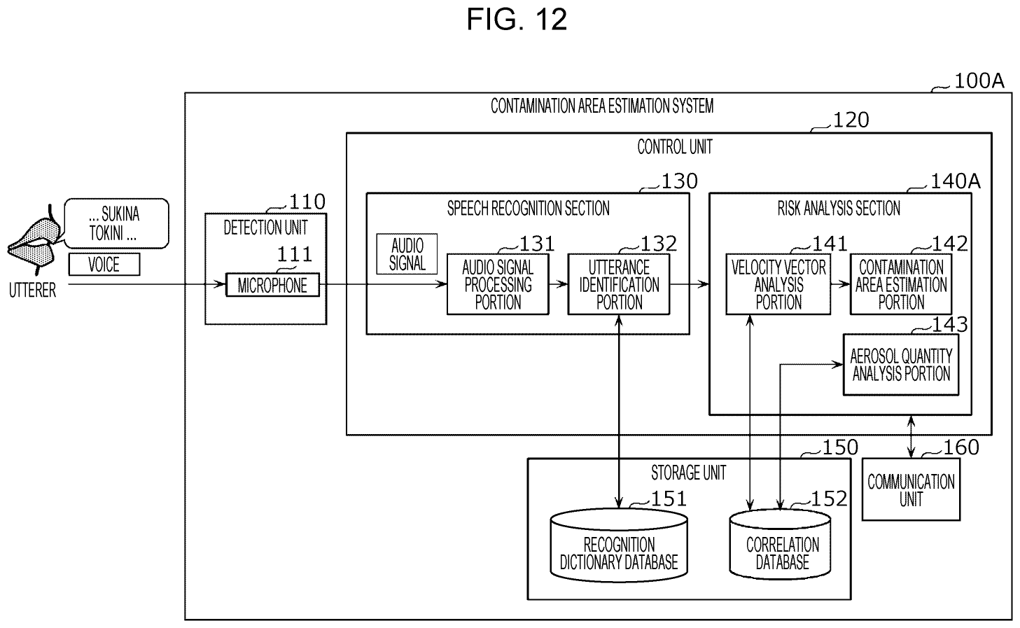

A contamination area estimation system 100A according to the first modification of the embodiment may also estimate, for each of speech sounds, the number of aerosol particles released from an utterer. FIG. 12 is a block diagram illustrating an example of the configuration of the contamination area estimation system according to the first modification of the embodiment.

As illustrated in FIG. 12, a risk analysis section 140A of a control unit 120A of the contamination area estimation system 100A also includes an aerosol quantity analysis portion 143 unlike the risk analysis section 140 according to the embodiment.

The aerosol quantity analysis portion 143 estimates the number of aerosol particles for each of monosyllables included in a result of utterance identification using the correlation database 152 stored in the storage unit 150. The aerosol quantity analysis portion 143 estimates, on the basis of second correlations, the number of aerosol particles from levels of sound pressure detected from speech sounds included in a voice detected by the detection unit 110. For example, the aerosol quantity analysis portion 143 estimates the number of aerosol particles caused when an utterer has uttered monosyllables included in a result of utterance identification performed by the utterance identification portion 132 by reading second correlations corresponding to the monosyllables from the correlation database 152 and identifying the number of aerosol particles corresponding, in the read second correlation, to detected levels of sound pressure of the monosyllable. The aerosol quantity analysis portion 143 adds up the number of aerosol particles for the monosyllables and sequentially calculates a total number of aerosol particles caused when speech sounds included in an audio signal have been uttered. The aerosol quantity analysis portion 143 may estimate a risk of viral infection on the basis of an obtained total value.

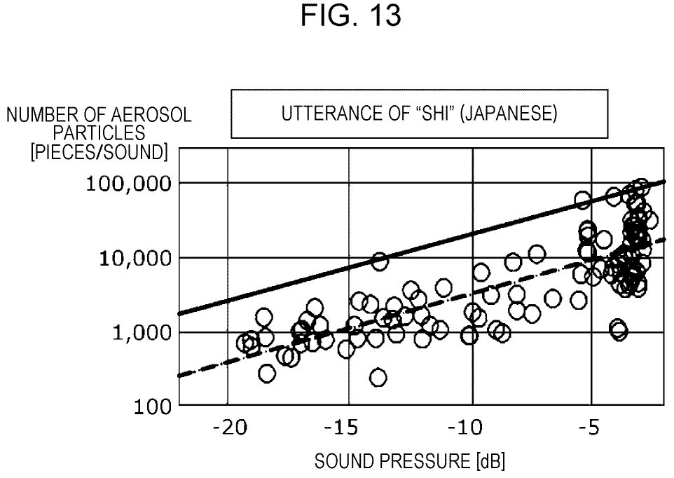

The correlation database 152 includes second correlations indicating relationships between speech sounds and the number of aerosol particles released from an utterer when the utterer utters the speech sounds. The second correlations indicate, as in FIG. 13, for example, sound pressure dependence of the number of aerosol particles released when monosyllables in speech sounds are uttered. FIG. 13 is a diagram illustrating results of measurement of sound pressure dependence of the number of aerosol particles released when a monosyllable “shi” (Japanese) is uttered once. The correlation database 152 thus includes second correlations between levels of sound pressure at times when an utterer has uttered monosyllables and the number of aerosol particles released from the utterer.

The correlation database 152 need not necessarily include second correlations regarding all monosyllables in speech sounds, and may include only second correlations regarding one or more monosyllables in certain speech sounds. In this case, the aerosol quantity analysis portion 143 may estimate the number of aerosol particles caused by monosyllables uttered by the utterer only for the certain speech sounds.

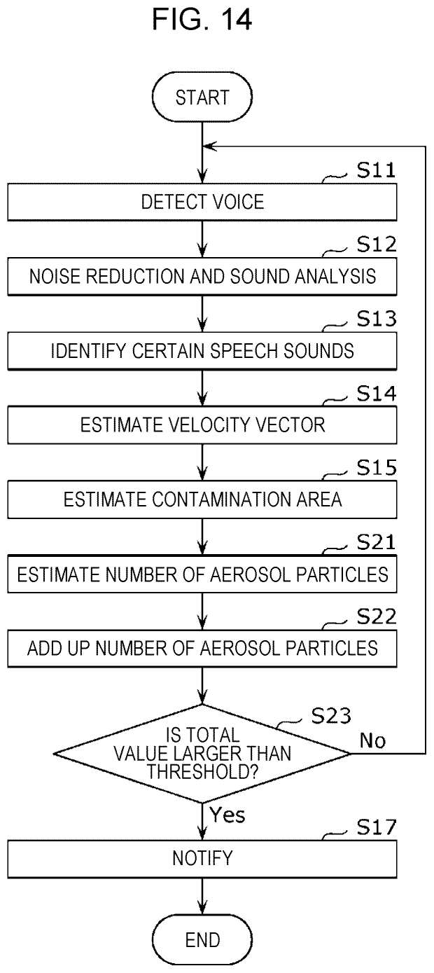

Next, an operation performed by the contamination area estimation system 100A according to the first modification of the embodiment will be described with reference to FIG. 14. FIG. 14 is a flowchart illustrating an example of the operation performed by the contamination area estimation system according to the first modification of the embodiment.

First, steps S11 to S15 are the same as those described in the embodiment, and description thereof is omitted.

Next, the aerosol quantity analysis portion 143 of the risk analysis section 140 estimates the number of aerosol particles for each monosyllable included in a result of the utterance identification using the correlation database 152 (S21). The aerosol quantity analysis portion 143 estimates the number of aerosol particles caused when the utterer has uttered each monosyllable included in the result of the utterance identification performed by the utterance identification portion 132 by reading a second correlation corresponding to the monosyllable from the correlation database 152 and identifying the number of aerosol particles corresponding, in the read second correlation, to a level of sound pressure of the identified monosyllable.

Next, the aerosol quantity analysis portion 143 adds up the number of aerosol particles for each monosyllable estimated in step S21 (S22). The aerosol quantity analysis portion 143 stores a total value in the storage unit 150. As a result, the aerosol quantity analysis portion 143 can calculate the total value (cumulative value) by adding the number of aerosol particles estimated in step S22 to a total value calculated in a previous addition. If the previous total value is not stored in the storage unit 150, the total value is calculated with the previous total value assumed as 0.

Next, the aerosol quantity analysis portion 143 determines whether the total value of the number of aerosol particles is larger than a certain number of aerosol particles (S23).

If the total value of the number of aerosol particles is larger than the certain number of aerosol particles (YES in S23), the aerosol quantity analysis portion 143 issues a notification (S17). If the total value of the number of aerosol particles is smaller than or equal to the certain number of aerosol particles (NO in S23), the aerosol quantity analysis portion 143 returns to step S11. That is, after calculating the total value of the number of aerosol particles or if determining that a notification need not be issued, the aerosol quantity analysis portion 143 performs the same process again. The contamination area estimation system 100 may keep performing the same process even after a notification is issued.

After step S13, step S16, which follows steps S14 and S15, and steps S21 to S23 may be performed in parallel with each other. If a result of step S16 or S23 is YES, a notification may be issued in step S17, and if the result of step S16 or S23 is NO, the process may return to step S11.

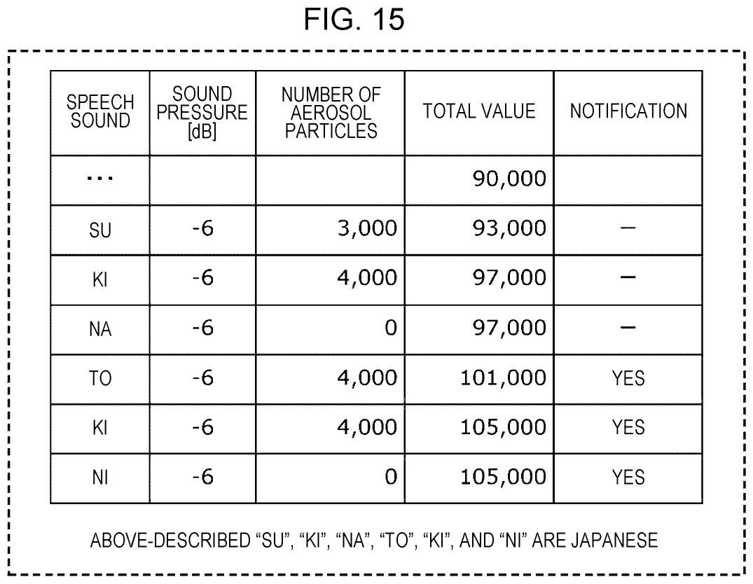

Next, a specific example of a process performed by the aerosol quantity analysis portion 143 of the risk analysis section 140 will be described with reference to FIG. 15. FIG. 15 is a diagram illustrating the specific example of the process performed by the aerosol quantity analysis portion. FIG. 15 is specifically a table indicating levels of sound pressure of monosyllables in speech sounds, the estimated number of aerosol particles, total values, and presence or absence of a notification.

The aerosol quantity analysis portion 143 calculates the number of aerosol particles for monosyllables in speech sounds identified by the utterance identification portion 132 by identifying the number of aerosol particles corresponding, in corresponding correlations, to levels of sound pressure of, among “su” (Japanese), “ki” (Japanese), “na” (Japanese), “to” (Japanese), “ki”, and “ni” (Japanese), which are the monosyllables in the speech sounds, for example, certain speech sounds “su” (Japanese), “ki” (Japanese), “to” (Japanese), and “ki” (Japanese). The aerosol quantity analysis portion 143 then adds up the calculated number of aerosol particles and, if a total value exceeds a certain number of aerosol particles (threshold), namely 100,000, for example, issues a notification. In this case, two different thresholds, namely 50,000 and 100,000, for example, may be set in order to issue notifications, and the aerosol quantity analysis portion 143 may issue a caution when the total value exceeds 50,000 and then issue a warning when the total value exceeds 100,000. The number of notifications is not limited to two, and three or more notifications may be issued, instead. After the notifications are issued, the total value stored in the storage unit 150 may be reset to 0.

### 1-5. Second Modification

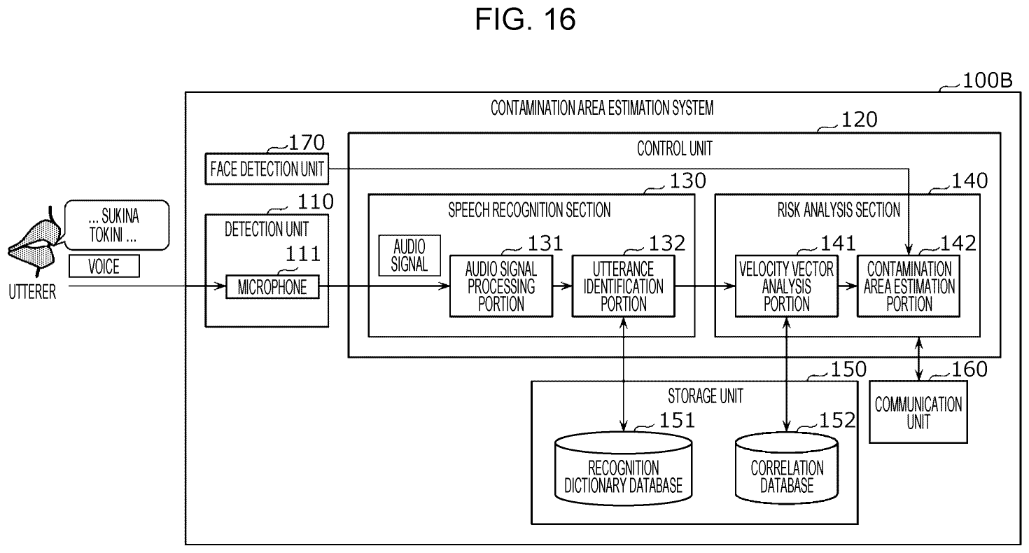

Next, a second modification of the embodiment will be described. FIG. 16 is a block diagram illustrating an example of the configuration of a contamination area estimation system according to the second modification of the embodiment.

A contamination area estimation system 100B according to the second modification of the embodiment may also include a face detection unit 170 that detects a position and a direction of an utterer's face. The face detection unit 170 may be, for example, a camera that captures an image of the utterer's face. The camera may be an RGB camera or an infrared camera.

The contamination area estimation portion 142 of the risk analysis section 140 of the control unit 120 may obtain the position and the direction of the utterer's face on the basis of a result of the detection performed by the face detection unit 170. More specifically, the contamination area estimation portion 142 may identify the position and the direction of the utterer's face (mouth) by analyzing an image captured by the camera, which is the face detection unit 170. The contamination area estimation portion 142 then estimates, as a contamination area, an area over which aerosol having a velocity vector is released from the mouth of an utterer Sp1 in the direction of the utterer's face with the position of the utterer's mouth obtained from the position and the direction of the utterer's face set as an origin.

As a result, since the face detection unit 170 detects a position and a direction of an utterer's face, the position and the direction of the utterer's face can be accurately identified, and a contamination area can be accurately estimated. In particular, the position and the direction of the utterer's face can be accurately identified using an image captured by the camera, and a contamination area can be accurately estimated.

### 1-6. Third Modification

The contamination area estimation system 100 according to the above embodiment may also include an infrared sensor that detects presence or absence of a person. The contamination area estimation portion 142 identifies the position and the direction of the face of the utterer Sp1 on the basis of object information and a result of the detection performed by the infrared sensor. As a result, presence of the utterer can be detected in a detection area of the infrared sensor. The contamination area estimation portion 142, therefore, can accurately detect the position of the utterer's face. When the infrared sensor is provided at a position from which the infrared sensor can detect presence or absence of a person in a space above a chair and is detecting presence of a person, for example, the contamination area estimation portion 142 can determine that the utterer Sp1 is likely to be sitting on the chair 201 in the space S1. The contamination area estimation portion 142, therefore, can accurately detect the position of the face of the utterer Sp1.

### 1-7. Fourth Modification

The contamination area estimation system 100 according to the above embodiment may also include an ultrasonic sensor that detects a space. The contamination area estimation portion 142 may identify a position of an object in a space on the basis of a result of the detection performed by the ultrasonic sensor. The contamination area estimation portion 142 may then identify a position and a direction of an utterer's face on the basis of the identified position of the object. For example, the contamination area estimation portion 142 may identify a position of the table 200 or the chair 201 in the space S1 on the basis of a result of the detection performed by the ultrasonic sensor. That is, the contamination area estimation portion 142 can identify, without obtaining object information, a position or a size of an object in the space S1 on the basis of a result of the detection performed by the ultrasonic sensor. Even when arrangement of objects or objects themselves have been changed in the space S1, therefore, a position of an utterer's face can be accurately identified.

### Other Embodiments

Although the contamination area estimation systems 100, 100A, and 100B according to the above embodiment and the modifications thereof issue a notification if, after the contamination area R1 is estimated, the area of the contamination area R1 is larger than the certain area (threshold) or a total value of the number of aerosol particles is larger than the certain number of aerosol particles (threshold), the present disclosure is not limited to the embodiment and the modifications.

For example, if the area of the contamination area R1 is larger than the certain area (threshold) or a total value is larger than the certain number of aerosol particles (threshold), the control unit 120 or 120A of the contamination area estimation system 100, 100A, or 100B may control a disinfection apparatus provided in or around a space where the detection unit 110 is provided in such a way as to spray a disinfectant solution or radiate ultraviolet light in the space. The control unit 120 or 120A may control the amount of disinfectant solution sprayed or the amount of ultraviolet light radiated (ultraviolet intensity or radiation time) in accordance with the area of the contamination area R1 or a total value. The control unit 120 or 120A may control the disinfection apparatus such that the amount of disinfectant solution sprayed or the amount of ultraviolet light radiated increases as the area of the contamination area R1 or the total value increases. The disinfection apparatus is provided outside the contamination area estimation system 100 or 100A. The disinfectant solution may be a liquid containing hypochlorous acid. If the area of the contamination area R1 or the number of aerosol particles with which it can be determined that the risk of infection is high is exceeded, therefore, the disinfectant solution can thus be sprayed or ultraviolet light can be radiated in the space. The disinfectant solution can be sprayed or ultraviolet light can be radiated in the space at appropriate times, thereby reducing the risk of infection.

If the area of the contamination area R1 is larger than the certain area (threshold) or a total value is larger than the certain number of aerosol particles (threshold), for example, the control unit 120 or 120A of the contamination area estimation system 100, 100A, or 100B may drive a ventilation fan or any other fan for ventilating a space where the detection unit 110 or 110A is provided. The ventilation may be performed not by the ventilation fan but by an air purifier, an air circulator fan, or the like. If the area of the contamination area R1 or the number of aerosol particles with which it can be determined that the risk of infection is high is exceeded, therefore, the space can be ventilated. The space can thus be effectively ventilated at appropriate times, thereby reducing the risk of infection.

The contamination area estimation system 100, 100A, or 100B according to the above embodiment or one of the modifications thereof may also include a body temperature measuring unit that measures body temperature of an utterer. The control unit 120 or 120A may determine whether the body temperature measured by the body temperature measuring unit exceeds a certain temperature and, if the body temperature exceeds the certain temperature, issue a warning. A warning, therefore, can be issued if it can be determined that the risk of infection is even higher.

Although identification of monosyllables in Japanese speech sounds has been described in the above embodiment and the modifications thereof, the scope of the present disclosure also includes modes where monosyllables in speech sounds in another language such as English or French are identified. Although the recognition dictionary database 151 of phonetic symbols is used for Japanese, a word-by-word recognition dictionary database and a correlation database including first correlations of corresponding velocity vectors and second correlations of the corresponding number of aerosol particles may be used for English or French. For Japanese, too, a word-by-word recognition dictionary database and a correlation database including first correlations of corresponding velocity vectors and second correlations of the corresponding number of aerosol particles may be used instead of the recognition dictionary database 151 of phonetic symbols.

Although the contamination area estimation systems 100, 100A, and 100B according to the above embodiment and the modifications thereof estimate the contamination area R1 on the top of the table 200, a three-dimensional area where aerosol released from an utterer is estimated to spread may be estimated as a contamination area using velocity vectors, instead.

Although the contamination area estimation systems 100, 100A, and 100B according to the above embodiment and the modifications thereof perform the estimation on the basis of a position and a direction of an utterer's face, the estimation may be performed on the basis of a position and a direction of an utterer's mouth, instead. In this case, a mouth detection unit that detects a position and a direction of an utterer's mouth may be included instead of the face detection unit 170.

Although the contamination area estimation system according to an embodiment of the present disclosure has been described, the present disclosure is not limited to this embodiment. For example, an aerosol reachable area estimation system that estimates not a contamination area but an area reachable by aerosol, for example, may be implemented, instead.

The processors included in the contamination area estimation system according to the above embodiment are achieved as a large-scale integration (LSI) chip, which is typically an integrated circuit. The processors may each be achieved as a chip, or some or all of the processors may be achieved as a chip.

An integrated circuit used is not limited to an LSI chip, and a dedicated circuit or a general-purpose processor may be used, instead. A field-programmable gate array (FPGA), where an LSI chip can be programmed after fabrication thereof, or a reconfigurable processor, where connections and settings of circuit cells inside an LSI chip can be reconfigured, may be used.

In the above embodiment, each component may be achieved by dedicated hardware or by executing a software program suitable for the component. Each component may be achieved by reading and executing a software program stored in a storage medium such as a hard disk or a semiconductor memory using a program execution unit such as a central processing unit (CPU) or a processor, instead.

The present disclosure may be achieved as a contamination area estimation method executed by the contamination area estimation system or the like.

Division of the functional blocks in the block diagrams is an example, and different functional blocks may be achieved as a single functional block, a single functional block may be divided into different functional blocks, or some functions may be transferred to other functional blocks. A single piece of hardware or software may process similar functions of different functional blocks in parallel or a time-division manner.

Order in which the steps of each flowchart are performed is an example for specifically describing the present disclosure, and may be changed. Some of the steps may be performed simultaneously (in parallel) with another step.

Although the contamination area estimation system and the contamination area estimation method according to one or more aspects have been described on the basis of an embodiment, the present disclosure is not limited to this embodiment. The scope of the present disclosure also includes modes achieved by modifying the above embodiment in ways conceivable by those skilled in the art and modes constructed by combining together components from different embodiments, insofar as the spirit of the present disclosure is not deviated from.

The present disclosure is effective as an aerosol reachable area estimation system, an aerosol reachable area estimation method, and the like capable of accurately estimating an area reachable by aerosol caused by an utterer when the utterer has uttered speech sounds.

What is claimed is:

1. An aerosol reachable area estimation system comprising:
a detector that detects a voice; and
a controller that estimates an area reachable by aerosol released to a space where an utterer who has emitted the voice detected by the detector exists from a speech sound included in the voice on a basis of a correlation between the speech sound and a velocity vector of aerosol released from the utterer when the utterer utters the speech sound and a position and a direction of the utterer's mouth.

2. The aerosol reachable area estimation system according to claim 1,
wherein the correlation is a correlation between way the speech sound is uttered and the velocity vector, and
wherein the controller (i) identifies the way the speech sound included in the voice is uttered and (ii) estimates the area on a basis of the velocity vector associated in the correlation with the identified way the speech sound is uttered and the position and the direction of the utterer's mouth.

3. The aerosol reachable area estimation system according to claim 2,
wherein the way the speech sound is uttered differs depending on a place of articulation used to utter the speech sound.

4. The aerosol reachable area estimation system according to claim 2,
wherein the way the speech sound is uttered differs depending on a manner of articulation used to utter the speech sound.

5. The aerosol reachable area estimation system according to claim 1,
wherein the correlation indicates that magnitude of the velocity vector becomes greater as the utterer's mouth opens smaller when the utterer utters the speech sound.

6. The aerosol reachable area estimation system according to claim 1,
wherein, in the correlation, a direction of a velocity vector associated with a speech sound uttered with a place of articulation such as teeth, an alveolar ridge, or a back of the alveolar ridge is more downward than a direction of a velocity vector associated with a speech sound uttered with another place of articulation.

7. The aerosol reachable area estimation system according to claim 1, further comprising:
a mouth detector that detects the position and the direction of the utterer's mouth,
wherein the controller obtains the position and the direction on a basis of a result of the detection performed by the mouth detector.

8. The aerosol reachable area estimation system according to claim 7,
wherein the mouth detector is a camera that captures an image of the utterer's mouth, and
wherein the controller analyzes the image captured by the camera and identifies the position and the direction.

9. The aerosol reachable area estimation system according to claim 1, further comprising:
an obtainer that obtains object information indicating arrangement of an object in the space where the utterer exists,
wherein the controller identifies the position and the direction on a basis of the object information.

10. The aerosol reachable area estimation system according to claim 9, further comprising:
an infrared sensor that detects presence or absence of a person,
wherein the controller identifies the position and the direction on a basis of the object information and a result of the detection performed by the infrared sensor.

11. The aerosol reachable area estimation system according to claim 1, further comprising:
an ultrasonic sensor that detects the space,
wherein the controller (i) identifies a position of an object in the space on a basis of a result of the detection performed by the ultrasonic sensor and (ii) identifies the position and the direction on a basis of the identified position of the object.

12. The aerosol reachable area estimation system according to claim 1,
wherein the controller notifies of the estimated area.

13. The aerosol reachable area estimation system according to claim 12,
wherein the controller notifies of the estimated area using an apparatus outside the aerosol reachable area estimation system.

14. The aerosol reachable area estimation system according to claim 13,
wherein the apparatus outside the aerosol reachable area estimation system is a display apparatus provided in the space or a mobile terminal owned by the utterer.

15. The aerosol reachable area estimation system according to claim 1,
wherein the controller sprays a disinfectant solution or radiates ultraviolet light onto the estimated area using the apparatus outside the aerosol reachable area estimation system.

16. The aerosol reachable area estimation system according to claim 1,
wherein the controller ventilates the space including the estimated area.

17. An aerosol reachable area estimation method comprising:
detecting a voice; and
estimating an area reachable by aerosol released to a space where an utterer who has emitted the voice detected in the detecting exists from a speech sound included in the voice on a basis of a correlation between the speech sound and a velocity vector of aerosol released from the utterer when the utterer utters the speech sound and a position and a direction of the utterer's mouth.

18. A non-transitory computer-readable recording medium storing a program causing a computer to execute the aerosol reachable area estimation method according to claim 17.

19. An aerosol reachable area estimation system comprising:
a detector that detects a voice including a speech sound uttered by an utterer, the utter uttering the speech sound with releasing aerosol; and
a controller that estimates an area reachable by the aerosol on a basis of first information, a position and a direction of the utterer's mouth, and the speech sound,
wherein the first information indicates that an i-th speech sound corresponds to an i-th velocity vector (i=1 to n, where n is an integer larger than or equal to 1), the i-th velocity vector being a velocity vector of an i-th aerosol released from a person uttering the i-th speech sound.
